(19) United States
(12) Patent Application Publication
(10) Pub. No.: US 2026/0029363 A1
(43) Pub. Date: Jan. 29, 2026

(54) DEVICE AND METHOD FOR TESTING AN ELECTROCHEMICAL SENSOR

(71) Applicant: Dräger Safety AG & Co. KGaA, Lübeck (DE)

(72) Inventors: Doreen Gimbel, Lübeck (DE); Malte Baesler, Lübeck (DE)

(21) Appl. No.: 19/035,463

(22) Filed: Jan. 23, 2025

(30) Foreign Application Priority Data
Jan. 23, 2024 (DE) 102024101872.0

(51) Int. Cl. G01N 27/27; G01N 27/407; G01N 33/00

(52) U.S. Cl. CPC G01N 27/27; G01N 27/407; G01N 33/007

(57) ABSTRACT

The disclosure relates to a device and method for automatically testing an electrochemical sensor. The electrochemical sensor is designed in the manner of a fuel cell and comprises a measuring chamber, two electrodes and an ionically conductive electrolyte between the two electrodes. A target gas to be detected causes an electrochemical reaction in the measuring chamber. The reaction causes an electric current to flow. A parameter of the flowing electrical current correlates with the sought target gas concentration. A current intensity curve [I(t)] is determined, which is the time course of the intensity of the current (I) caused to flow by the electrochemical reaction. A plurality of parameters [T₇₀, T_{70,35}, T_{70,70}, T_{standard}] for the current intensity curve [I(t)] are determined. A measure of the current humidity of the electrolyte is determined. The parameters [T₇₀, T_{70,35}, T_{70,70}, T_{standard}] determined for the current intensity curve [I(t)] are used for this purpose.

### CROSS-REFERENCE TO RELATED APPLICATIONS

This application claims the priority of German Patent Application No. 102024101872.0, filed on Jan. 23, 2024, the entire contents of which being fully incorporated herein by reference.

### DESCRIPTION

The disclosure relates to a test device and to a test method for automatically testing an electrochemical sensor. The disclosure further relates to an analyzer and to a measuring method for determining the concentration of a given target gas in a gas sample, wherein the analyzer comprises the test device and the measuring method comprises the steps of the test method.

Various analyzers having an electrochemical sensor have become known and are used to test the blood alcohol level of a subject. It is well-known that a breath sample provided by the subject will contain breath alcohol if alcohol is present in the subject's blood circulation. The analyzer takes a portion of the breath sample given as a gas sample, and the electrochemical sensor determines the breath alcohol content in the gas sample.

An object of the disclosure is to provide a test device and a test method for testing an electrochemical sensor and thereby increasing the reliability of the electrochemical sensor in comparison with known devices and methods. Furthermore, an object of the disclosure is to provide an analyzer and a measuring method for measuring the concentration of a given target gas in a gas sample, wherein the analyzer and the measuring method are intended to be more reliable than known analyzers and measuring methods.

The object is achieved by a test device having the features of claim 1 and by a test method having the features of claim 9 as well as by an analyzer having the features of claim 6 and by a measuring method having the features of claim 11.

The electrochemical sensor to be tested comprises a measuring chamber, two electrodes, and an ionically conductive electrolyte between the two electrodes. The measuring chamber can take up (receive, hold) a gas sample. The gas sample originates from a spatial region to be monitored. If the spatial region contains a target gas with a sufficient concentration (share), the target gas will usually also be present in the gas sample. One electrode is the measuring electrode, and the other electrode is the counter-electrode. Optionally, the electrochemical sensor also includes a reference electrode.

The electrolyte has a humidity that varies or can vary over time. In one embodiment, the electrolyte humidity is understood to be the amount of substance or the molar concentration, also called molarity. The amount of substance or molar concentration refers to parts that are mobile in a liquid solution and therefore electrically conductive, related to the volume of electrolyte. The higher the amount of substance or the molar concentration is, the lower is the electrolyte humidity. The amount of substance is measured in [mol] or [mmol], the molar concentration for example in [mol/l]. Typically, the electrolyte comprises water or another solvent. The electrolyte humidity is then understood to be the molar concentration of the solvent, measured in [mol/l].

The electrochemical sensor is designed like a fuel cell as follows: A combustible target gas in the measuring chamber causes an electrochemical reaction. The electrochemical reaction causes an electrical current to flow between the two electrodes. A measurable detection variable, which depends on the flowing electrical current, correlates with the sought concentration of the target gas in the measuring chamber, more precisely: with the target gas concentration in a gas sample in the measuring chamber. In particular, the detection variable is the entire electrical charge Q which flows in the course of the induced electrochemical reaction.

Note: In the following, repeatedly the formulation that a sensor measures a physical variable, for example the target gas concentration, is used. This means the following: The sensor directly measures the physical variable or at least one other variable that correlates with the sought physical variable and is therefore an indicator for the sought physical variable, for example the electrical charge Q or another detection variable for the target gas concentration. The measurement provides at least one value for the sought physical variable.

A plurality of curve parameters is given (specified). Each given curve parameter is a parameter of a time course (time curve) of a current intensity. Each curve parameter characterizes the time course of the current intensity at least when-according to the time course—the current intensity is initially increasing, reaches a maximum, and then decreases again. Typically, the intensity of the current flowing between the electrodes of an electrochemical sensor comprises such a time course that initially increases and then decreases again, namely at least when a gas sample in the measuring chamber has a sufficiently high concentration of a combustible target gas and the measuring chamber is separated from the environment during measurement in a sufficiently fluid-tight manner.

Furthermore, a computer-evaluable humidity function is given. The humidity function describes the humidity of the electrolyte as a function of the given curve parameters. Preferably, the humidity function is determined empirically beforehand using a sample with a plurality of sample elements wherein every sample element comprises a respective value for every curve parameter and a value for the actual electrolyte humidity.

The test device according to the disclosure comprises a signal-processing control unit. The control unit is configured to determine an actual current intensity curve (current measurement curve). The current intensity curve is the time course of the intensity of the electric current caused to flow by the induced electrochemical reaction. Of course, the actual current intensity curve can usually only be determined approximately.

Preferably, the test device comprises a current intensity sensor. This current intensity sensor is configured to measure the intensity of the induced electrical current and to generate a signal. The current intensity signal contains information about the time course of the measured current intensity. The control unit is configured to receive the generated current intensity signal and thereby determine the current measurement curve.

The control unit is configured to determine at least approximately the current humidity of the electrolyte between the two electrodes of the electrochemical sensor. For this purpose, the control unit uses the actual current intensity curve determined. The way in which the control unit determines the electrolyte humidity according to the disclosure will be explained below.

As already mentioned, a plurality of (at least two) curve parameters are given. For each curve parameter, the control unit is configured to determine which value the curve parameter will assume for the actual current intensity curve determined. This determination provides a plurality of curve parameter values.

Furthermore, according to the disclosure, a computer-evaluable humidity function is given, wherein the humidity function describes the electrolyte humidity as a function of the curve parameters. The humidity function is preferably stored in a data memory of the test device, to which memory the control unit has read access, at least from time to time, or is a component of a program executed or executable by the control unit. The control unit is configured to apply the humidity function to the curve parameter values determined. This application provides at least an approximation of the actual current electrolyte humidity.

The disclosure relates to an electrochemical sensor. In particular, an electrochemical sensor has the following advantage over an alternative sensor which is also configured to measure the concentration of a combustible target gas in a gas sample: An analyzer having an electrochemical sensor generally consumes less electrical energy than an analyzer having a different sensor, in particular less than an analyzer with a photoelectric or an oxidizing sensor. This is particularly advantageous if the analyzer comprises its own power supply unit and has no, or at least no permanent, connection to a stationary power grid.

In one possible application, the target gas is breath alcohol, which may be contained in a subject's breath sample. Breath alcohol absorbs electromagnetic radiation in a wavelength range around 9.5 μm. Especially in the case of this application, an electrochemical sensor is often cheaper to manufacture than a photoelectric sensor for this wavelength range.

A target gas, which is present as part of a gas sample in the measuring chamber, causes an electrochemical reaction at the electrochemical sensor. This electrochemical reaction causes an electric current to flow between the two electrodes. A measurable detection variable of a time course of this electric current, in particular the electrical charge Q, can be used as an indicator for the concentration of the target gas in the gas sample.

However, the detection variable that depends on the current intensity curve, in particular the electrical charge Q, depends not only on the sought target gas concentration but also on the current humidity of the electrolyte between the two electrodes. The electrolyte humidity can change over time, especially if the analyzer is stored for a relatively long time period or if the measuring chamber and thus the electrochemical sensor of the analyzer is fluidically connected to the environment.

At least from time to time, such a fluidic connection to the environment must be established so that the measuring chamber can take up a gas sample to be tested. In addition, such a fluidic connection is often even present, or at least may be present, in a situation in which no gas sample flows into the measuring chamber. For example, a fluidic connection between the measuring chamber and the environment cannot be closed due to the lack of a closure element, or the fluid connection is not closed. Or a valve or other closure element is not completely fluid-tight. In addition, it is possible that due to unavoidable material tolerances and gaps the measuring chamber is not ideally separated from the environment in a fluid-tight manner.

If the analyzer is stored for a relatively long period of time in an environment with relatively low air humidity, liquid from the electrolyte may evaporate through unavoidable gaps and slots in the analyzer, causing the electrolyte humidity to decrease. Conversely, if stored in an environment with relatively high air humidity, the electrolyte humidity may increase. An aqueous solution is often used as the electrolyte, for example a solution of sulfuric acid in water. Sulfuric acid and other potential substances in the electrolyte are highly hygroscopic. When the measuring chamber is fluidically connected to the environment, often a humidity equilibrium occurs between the electrolyte inside an apparatus comprising the electrochemical sensor and the environment. This humidity equilibrium can also occur due to unavoidable gaps during relatively long-term storage. The process of a humidity equilibrium being established often leads to a relatively large change in volume of the electrolyte.

In internal experiments, the inventors used an aqueous solution of sulfuric acid as the electrolyte and found the following relationship between the humidity in the environment and the sulfuric acid concentration at an ambient temperature of 20° C.:

| Ambient humidity | Sulfuric acid |
|---|---|
| in [%] | concentration in [mol/l] |
| 10 | 12 |
| 70 | 4 |
| 90 | 2 |

Accordingly, the higher the ambient humidity is, the greater is the concentration of the solvent water.

According to a reasonable definition, electrolyte humidity is the water concentration in the electrolyte. According to this definition, the lower the air humidity is, the lower the electrolyte humidity.

The disclosure makes it possible to determine at least approximately the current electrolyte humidity. In many cases, knowledge of the current electrolyte humidity enables the following:
The effect of the electrolyte humidity on a sensor measurement result can be at least approximately compensated for by way of calculation. In particular, the effect of the electrolyte humidity on the electrical charge Q or on the other detection variable that correlates with the desired target gas concentration can be compensated for by way of calculation. This increases the reliability and/or measurement accuracy of the electrochemical sensor compared to a design in which the changing electrolyte humidity is not taken into account.
Knowledge of the electrolyte humidity and in particular knowledge of its time course makes it possible to test the electrolyte automatically. If the electrolyte humidity is outside a given target value range for a sufficiently long time, a notification can be generated that the electrochemical sensor needs to be tested and/or that the electrolyte must be replaced or replenished.

The disclosure generally does not require an additional sensor for determining the electrolyte humidity. A sensor for measuring the intensity of the current flowing between the two electrodes is usually present anyway. Such a sensor is in fact usually required for determining the target gas concentration. Often, the disclosure can be implemented by correspondingly modifying the software executed by a control unit.

According to the disclosure, a humidity function is specified in a computer-evaluable form. The humidity function describes the electrolyte humidity as a function of the given curve parameters. Preferably, the humidity function is determined empirically beforehand. To determine the humidity function, a sample with a plurality of sample elements is generated and evaluated. In one embodiment, each sample element comprises, on the one hand, a value for the actual electrolyte humidity and a value for a target gas concentration and, on the other hand, the actual current intensity curve that the electrochemical sensor provides at this electrolyte humidity and this target gas concentration. In another embodiment, each sample element comprises, on the one hand, a value for the electrolyte humidity and the target gas concentration and, on the other hand, the value assumed by a current intensity curve for each given curve parameter, wherein this current intensity curve was determined at this electrolyte humidity and this target gas concentration.

The actual current intensity curve is influenced by the electrolyte humidity and usually by other factors, in particular the ambient temperature. According to the disclosure, however, the electrolyte humidity is not determined directly on the basis of the current intensity curve but on the basis of the humidity function and the curve parameter values.

It is possible that the test device according to the disclosure comprises a temperature sensor which can measure the ambient temperature or is designed to receive a signal from a temperature sensor spaced apart therefrom, wherein the received signal contains information about a temperature measured in the environment of the electrochemical sensor.

However, the inventors have found in internal tests that the actual electrolyte humidity correlates well with the values of the curve parameters. By a suitable choice of the humidity function, the influence of the ambient temperature and of other influencing factors can be eliminated or at least significantly reduced. For this reason, neither the ambient temperature nor other potentially influencing variables need to be measured in order to determine the electrolyte humidity. Other variable quantities often have a significantly smaller influence on the electrolyte humidity than a humidity value calculated by applying the humidity function to the curve parameter values. For this reason, in conjunction with the humidity function, the curve parameters make it possible to determine the electrolyte humidity relatively well. The electrolyte humidity determined according to the disclosure generally still differs from the actual electrolyte humidity.

As a rule, the current humidity of an electrolyte can only be directly measured in an analyzer with a great deal of effort. According to the disclosure, the control unit indirectly determines the current humidity of the electrolyte. For this determination, the control unit uses a plurality of curve parameters of the actual current intensity curve that has been determined. Embodiments of the disclosure define different possible curve parameters which can be used to determine the electrolyte humidity.

In one embodiment, the at least one curve parameter is the time period (temporal duration) between the following two timepoints:
the start of measurement (t=0), and
either the earliest or the latest time point at which the current curve becomes equal to x₁% of the maximum current.

A curve parameter can also be the time period between the following time points:
either the earliest or the latest time point at which the current intensity curve is equal to x₁% of the maximum current intensity, and
either the earliest or the latest time point at which the current intensity curve is equal to x₂% of the maximum current intensity.

Here, x₁ and x₂ are two percentages. The two percentages may correspond or differ. If at the two time points the current intensity is x₁% or x₂% for the first occurrence or x₁% or x₂% for the last occurrence, these two percentages x₁% or x₂% will of course differ from one another.

In another embodiment, the at least one curve parameter is the electrical charge that flows between the following two time points:
the start of measurement (t=0), and
either the earliest or the latest time point at which the current intensity curve is equal to x₁% of the maximum current intensity. The maximum current intensity is the maximal value of the actual time course of the current intensity.

The at least one curve parameter can also be the electrical charge that flows between the following two time points:
either the earliest or the latest time point at which the current intensity curve is equal to x₁% of the maximum current intensity, and
either the earliest or the latest time point at which the current intensity curve is equal to x₂% of the maximum current intensity.

These two curve parameters are preferably calculated as the area under the curve describing the current intensity curve between these two time points.

In a further embodiment, the at least one curve parameter is a secant, in particular the slope of the secant, between two points on the curve that describes the current intensity curve. The corresponding x value of these two curve points is a specific time point, in particular the start of the measurement or the earliest or latest time point at which the current intensity curve is equal to x₁% or x₂% of the maximum current intensity. The corresponding y value of these two points is the associated current intensity at that time point. A special case of a slope of a secant is the slope of the curve (of the tangent) at a point.

In a further embodiment, a curve parameter is the time point at which the curve describing the current intensity curve has an inflection point.

According to the disclosure, the control unit determines the current electrolyte humidity by using a plurality of curve parameters (more precisely: a plurality of parameter values) of the current intensity curve, whereby various possible curve parameters having been described above. According to the disclosure, in order to determine the electrolyte humidity, the control unit applies the given computer-evaluable humidity function to the values determined for the curve parameters used.

In an exemplary embodiment, the predetermined humidity function is a quotient with a numerator and a denominator. The numerator is a numerator humidity function, and the denominator is a denominator humidity function. At least one curve parameter occurs in both the numerator and the denominator. In one embodiment, the numerator humidity function is a weighted average or a median over at least two curve parameters.

In internal tests, the inventors found that when using a humidity function in the form of such a quotient, the influence of other factors, in particular of the ambient temperature, is compensated for relatively effectively. More specifically: The values of the curve parameters have relatively little dependence on other influencing factors. If, therefore, the humidity function has the form of such a quotient, it is in many cases not necessary to measure the ambient temperature and additionally make the humidity function dependent on the measured ambient temperature. This design thus eliminates the need for a sensor for measuring the ambient temperature.

As a rule, the actual current intensity curve has the following form: The current intensity increases to a maximum (hereinafter: maximum current intensity) and then decreases again. In one embodiment, a curve parameter that occurs in the denominator humidity function is the time period between the following two time points:
the earliest time point at which the current intensity curve is equal to x₁% of the maximum current intensity, and
the latest time point at which the current intensity curve is equal to x₂% of the maximum current.

x₁ and x₂ are two percentages. These two percentages x₁ and x₂ may coincide or differ from each other. Preferably, both x₁ and x₂ lie between 10% and 30%, particularly preferably between 15% and 25%.

In this implementation, the actual current intensity curve is standardized over the temporal extent of the curve. This embodiment compensates particularly well for the influence of the ambient temperature.

In one embodiment, the humidity function specifies that a weighted average or a median over the curve parameters used is used as the electrolyte humidity. In another embodiment, the humidity function specifies that a quotient is used as the electrolyte humidity. The numerator of this quotient is a curve parameter or a weighted average or a median of a plurality of curve parameters. The denominator is also a curve parameter or a weighted average or a median of a plurality of curve parameters.

The or each weighted average just described is calculated using given weighting factors. Preferably, these weighting factors are determined empirically beforehand by generating and using a sample. Each sample element comprises a measured or given value for the electrolyte humidity, a measured or given value for the target gas concentration and the current intensity curve resulting from these two values.

In one embodiment, the control unit can receive a signal containing information about a temperature measured in the environment of the electrochemical sensor. This ambient temperature is measured by a temperature sensor. The temperature sensor can be a component of the test device according to the disclosure. The given humidity function depends on at least one curve parameter and additionally on the ambient temperature.

The disclosure further relates to an analyzer and to a measuring method which can measure the concentration of a given target gas in a gas sample. The analyzer comprises a measuring chamber. The measuring chamber can take up (hold) the gas sample. The analyzer further comprises an electrochemical sensor and a test device according to the disclosure. The electrochemical sensor is constructed as described above with reference to the test device. The measuring method is carried out using such an analyzer. When carrying out the measuring method, the steps of the test method according to the disclosure are carried out.

Thanks to the test device, the analyzer is therefore configured to test itself. It is possible, but thanks to the disclosure not necessary, to use an external test apparatus. In many cases, this makes it possible to determine relatively quickly whether the electrolyte humidity in the analyzer is too low or too high. It is also possible that an external test apparatus according to the disclosure determines the electrolyte humidity and the determined electrolyte humidity is transmitted to the analyzer and is used by the analyzer.

The steps according to the disclosure by which the current electrolyte humidity is determined are preferably initiated (triggered) by determining that the target gas concentration lies outside a given concentration value range, in particular above a given lower concentration limit. For example, the electrolyte humidity is determined again each time the target gas concentration lies above the concentration limit, or every n-th time, where n is a given number greater than or equal to 2. It is also possible to determine the current electrolyte humidity after every n-th use of the analyzer, where n>=1 is a given number.

Embodiments of the test device according to the disclosure are also embodiments of the analyzer according to the disclosure. Embodiments of the test method according to the disclosure are also embodiments of the measuring method according to the disclosure.

In one embodiment, a concentration function is given in a computer-evaluable form. The concentration function describes the target gas concentration as a function of the detection variable on the one hand and of the electrolyte humidity on the other, optionally further depending on a measured ambient condition, e.g. the temperature. The analyzer according to the disclosure is configured to measure the target gas concentration as follows: The analyzer applies the concentration function to the measured detection variable and to the determined electrolyte humidity, optionally further on the measured ambient condition. In many cases, the following assumption is justified and can be applied when setting up the concentration function: At a constant actual target gas concentration, the detection variable has a linear dependence on the electrolyte humidity, specifically preferably such that the lower the electrolyte humidity is the smaller is the detection variable. It is also possible to empirically determine a functional relationship beforehand, wherein the determined functional relationship describes the detection variable as a function of the target gas concentration and the electrolyte humidity.

According to the disclosure, a humidity function is formulated in advance and used during use of the test device. In the previous paragraph, an implementation was described in which a concentration function is formulated in advance. This concentration function is used when using the analyzer. In general, one sample is required for each application. For example, the sample is used to determine a value for at least one model parameter of the humidity function and/or for at least one model parameter of the concentration function. Alternatively, a learning method is used to set up the humidity function and/or the concentration function.

The embodiment described below eliminates the need to measure the actual electrolyte humidity for this purpose. Instead, use is made of the following fact: At least under laboratory conditions, a certain ambient humidity leads to a certain electrolyte humidity, provided that a sufficient fluid connection between the electrochemical sensor with the electrolyte and the environment is established for a sufficiently long time and the ambient humidity remains constant. Under laboratory conditions, a certain known ambient humidity can usually be established sufficiently reliably. The higher the ambient humidity, the higher the electrolyte humidity as well. Other influencing variables, especially temperature, have a much smaller effect on electrolyte humidity.

A range of values from a to b is specified, for example from 0 to 1 or from −1 to +1. The electrolyte humidity that occurs at the lowest possible ambient humidity, for example 0%, is coded with a, the electrolyte humidity at the highest possible ambient humidity, for example 100%, is coded with b. Any electrolyte humidity that results from an ambient humidity between the lowest possible and the highest possible value is coded with a value between a and b. It is not necessary to measure the real value actually assumed by the electrolyte humidity at a certain ambient humidity.

According to this embodiment, the humidity function according to the disclosure provides a value from the range from a to b for the desired electrolyte humidity. The concentration function does not feature the actual electrolyte humidity but the coded electrolyte humidity. When the analyzer is used, the coded electrolyte humidity, i.e. a value from the range from a to b, is determined according to the disclosure and is inserted into the concentration function.

The analyzer may be designed as a portable device which is configured to be held in a person's hand or attached to a person's protective clothing. In this embodiment, the analyzer preferably has its own power supply unit. In addition, the analyzer preferably has its own output unit on which the determined target gas concentration and/or a warning regarding a too high or too low target gas concentration is output, specifically in at least one form perceptible by a person. In one embodiment, this output unit also displays an electrolyte humidity that is too low or too high. The notifications are output visually, acoustically and/or haptically (through vibrations), for example.

The analyzer can also be configured as a stationary apparatus that is installed add a fixed installation location and can preferably be connected to a stationary power supply network. In this application, the analyzer preferably comprises a communication unit which is configured to transmit a notification containing a determined target gas concentration to a receiver spaced apart therefrom.

The target gas is preferably a combustible target gas, for example breath alcohol or methane or hydrogen or even an anesthetic. It is possible that the analyzer can determine the cumulated concentrations of a plurality of target gases.

In the following, the disclosure is described on the basis of an exemplary embodiment. In the drawings,

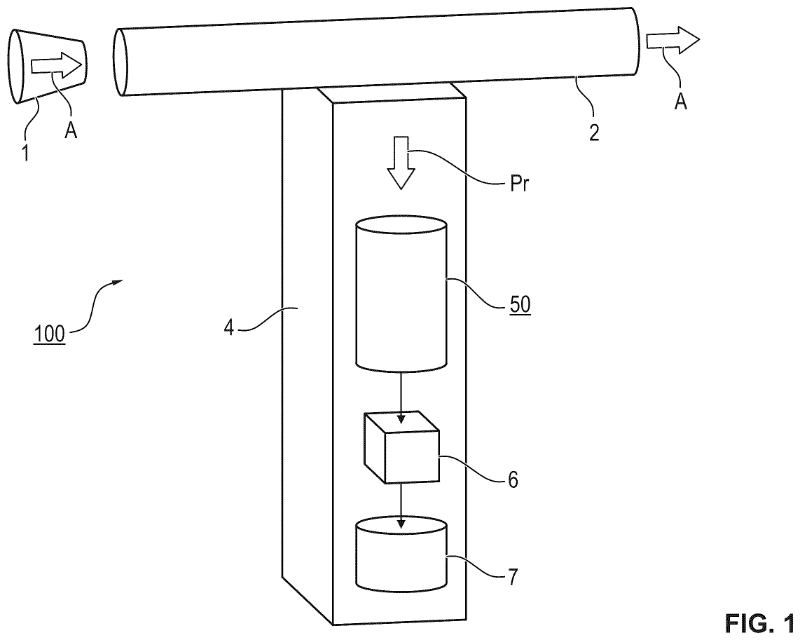

FIG. 1 schematically shows an analyzer with an electrochemical sensor;

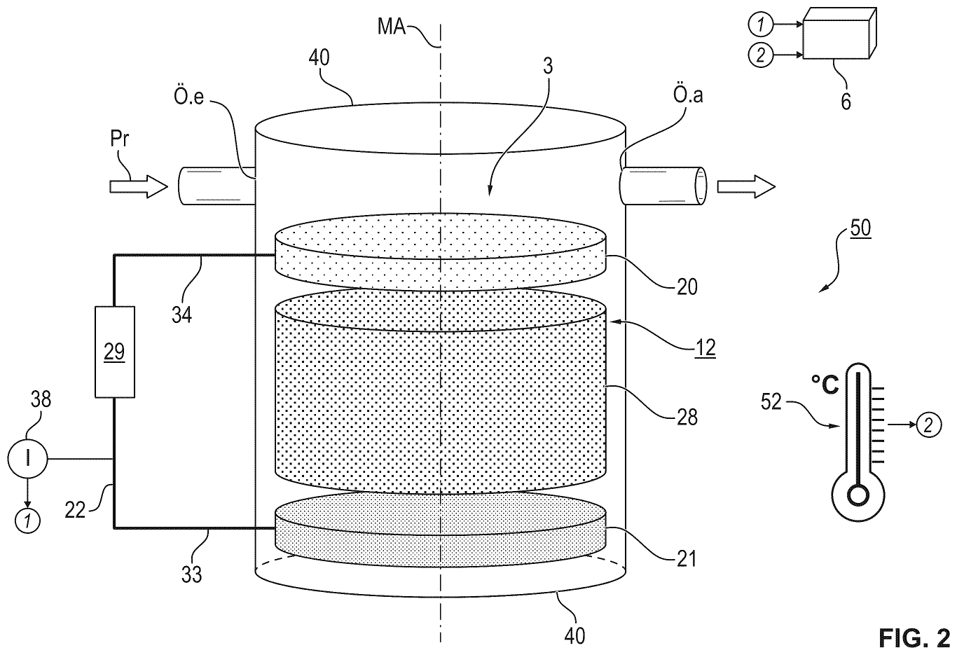

FIG. 2 schematically shows the mode of operation of an electrochemical sensor;

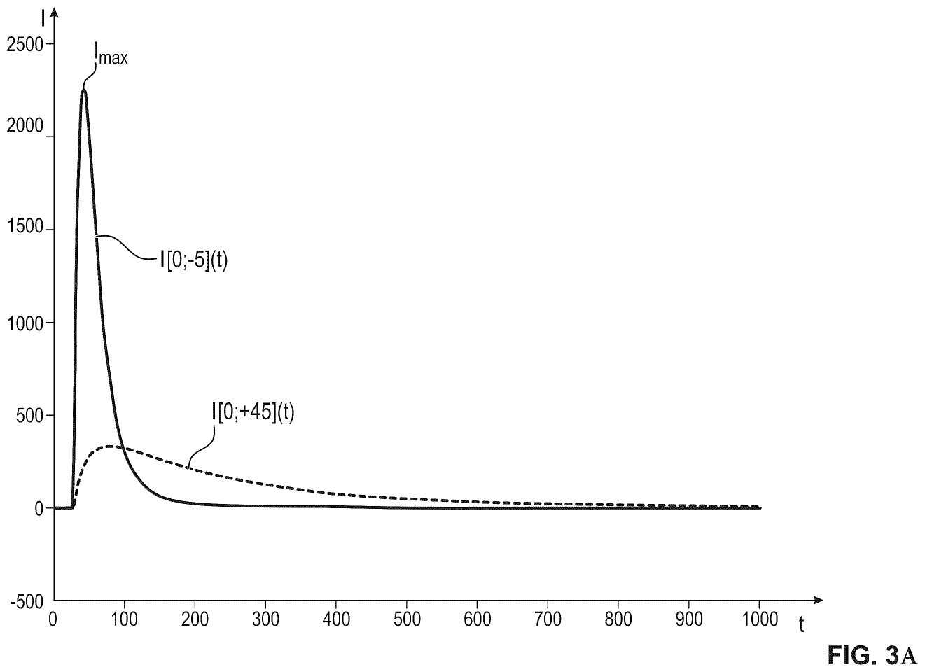

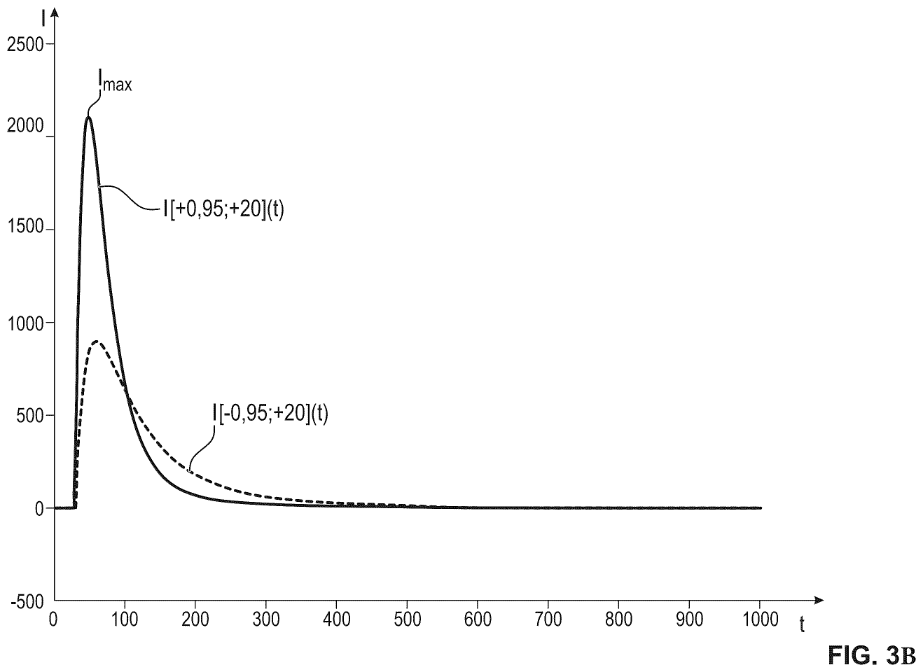

FIG. 3A shows the time course of the current intensity at different ambient temperatures and different electrolyte humidities;

FIG. 3B shows further examples of the time course of the current intensity at different ambient temperatures and different electrolyte humidities;

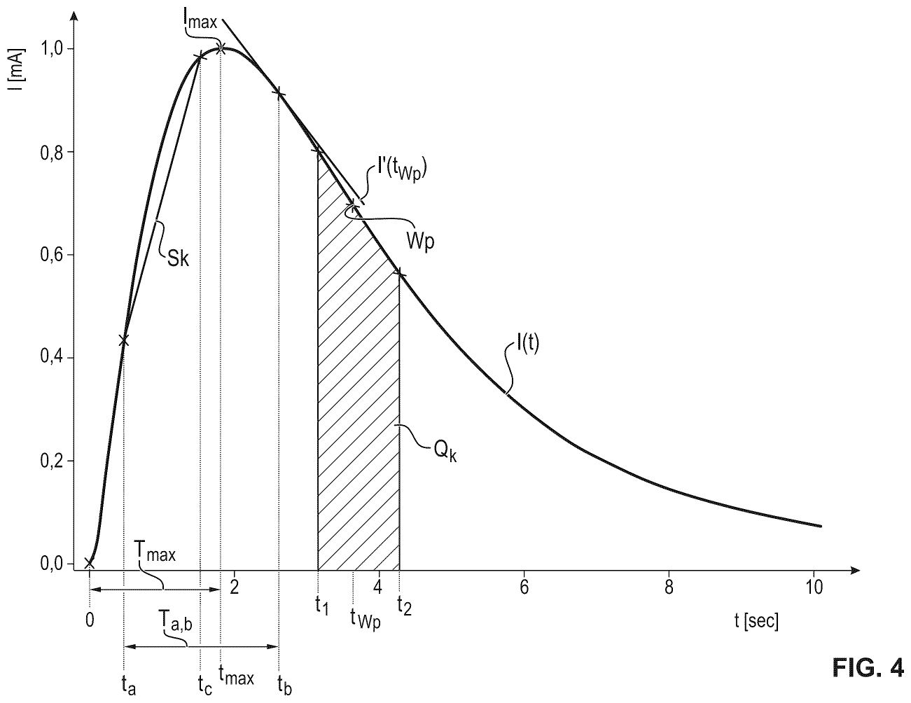

FIG. 4 shows the time course of the current intensity as well as a number of curve parameters of this time course;

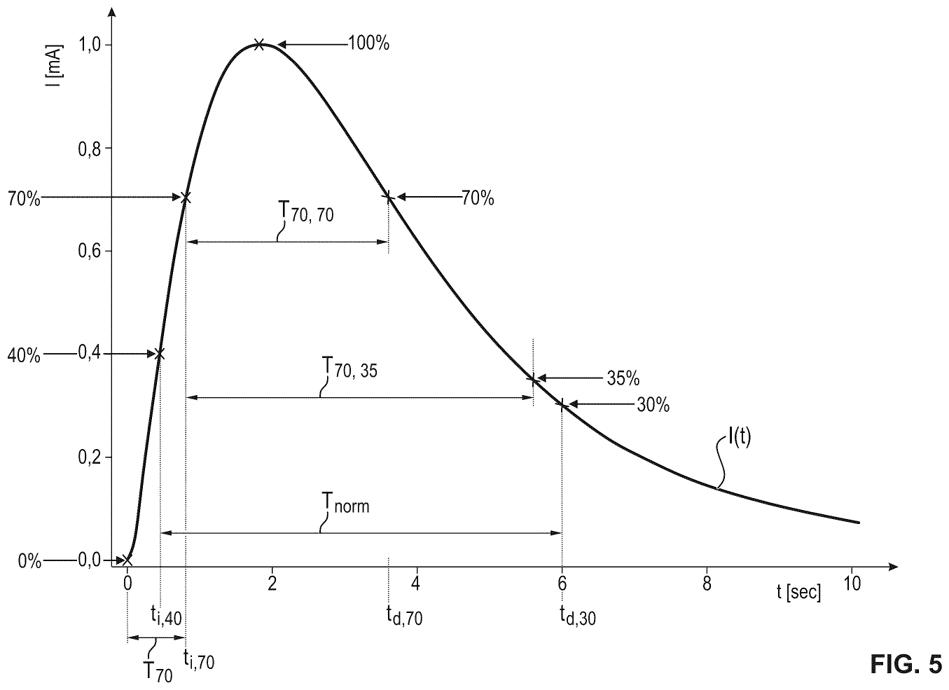

FIG. 5 shows the time course from FIG. 4 and some other time course parameters;

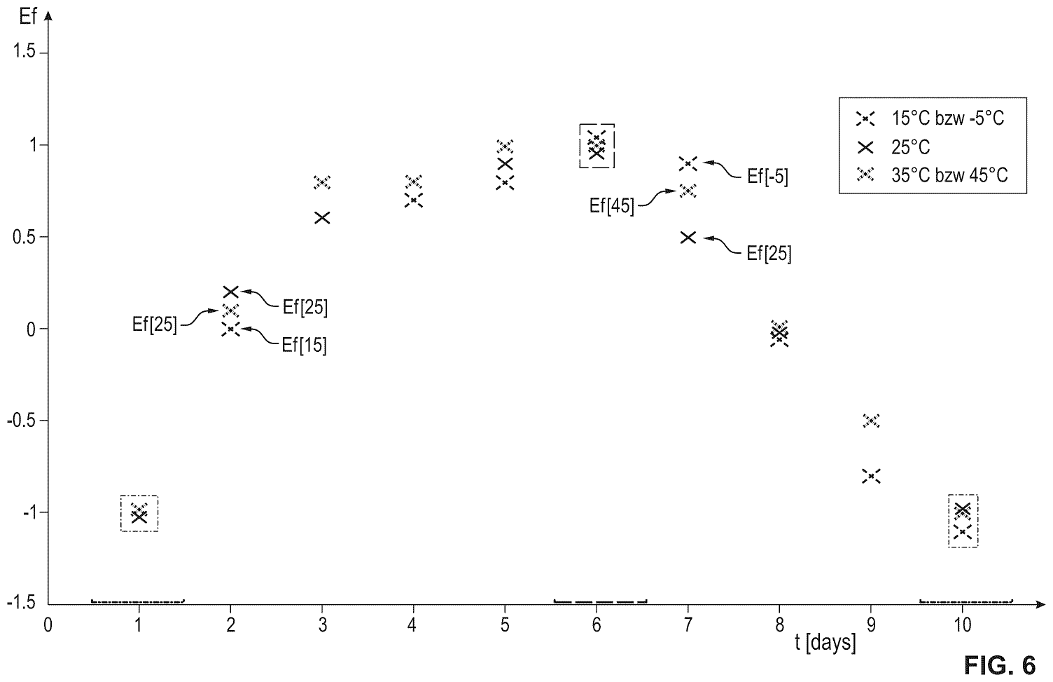

FIG. 6 shows exemplary measurement results.

The disclosure can be used for an analyzer which measures the concentration of at least one target gas in a gas mixture. In one application, the gas mixture is a breath sample from a subject and the target gas is breath alcohol. The aim is to determine whether or not alcohol is present in the subject's blood circulation. Alcohol in the blood circulation is known to lead to breath alcohol in a breath sample. In other applications, the gas mixture is ambient air, and the target gas is in one application a flammable (combustible) gas or a gas that is harmful to humans in some other way and in another application oxygen or an anesthetic.

FIG. 1 schematically shows an analyzer 100. The analyzer 100 comprises a housing 4 in which a sensor arrangement 50, a signal-processing control unit 6 and a data memory 7 are arranged. The sensor arrangement 50 comprises an electrochemical sensor and a measuring chamber. A person can hold the housing 4 in one hand. This person can be the subject to be tested or another person, such as a police officer. A tube 2 is attached, preferably detachably, to the housing 4. A funnel-shaped mouthpiece 1 can be connected to the tube 2.

The person holds the analyzer 100 in such a way that the mouthpiece 1 is in front of the mouth of a subject. The subject gives by exhaling a breath sample A into the mouthpiece 1. The breath sample A flows through tube 2. A portion of the breath sample A is diverted out of the tube 2 and flows into the measuring chamber as a measuring chamber sample Pr. Preferably, a suitable fluid delivery unit (not shown) draws in the measuring chamber sample Pr from the breath sample A and delivers it to the measuring chamber. The remainder of the breath sample A flows through tube 2 back into the environment.

The analyzer 100 thus comprises a measuring chamber which holds the measuring chamber sample Pr to be analyzed and a sensor which measures the concentration of the target gas in the gas sample. Various principles that such a sensor can apply have become known. An electrochemical sensor, as described below, has the advantage over other sensor principles in that it requires less electrical energy. This is particularly advantageous if the analyzer 100 is to be used without a permanent connection to a stationary power supply network and therefore comprises its own power supply unit.

The disclosure can be used to test such an electrochemical sensor. FIG. 2 schematically shows, by way of example, the mode of operation of an electrochemical sensor 12 as known from the prior art. The representation in FIG. 2 is not necessarily true to scale.

This electrochemical sensor 12 belongs to the sensor arrangement 50 from FIG. 1. The sensor 12 can analyze a measuring chamber sample Pr for a target gas and operates according to the principle of a fuel cell with the target gas as the fuel. The target gas is, for example, breath alcohol contained in the subject's breath sample A or an aldehyde produced during the oxidation of alcohol.

Reference sign 50 in FIG. 1 and FIG. 2 designates a sensor arrangement which comprises the core electrochemical sensor 12 and a wall 40 for a measuring chamber 3. The wall 40 surrounds the sensor 12 and the measuring chamber 3. In the embodiment shown, both the wall 40 and the sensor 12 are rotationally symmetrical to the same central axis MA, which axis lies in the plane of the drawing in FIG. 2. Of course, other geometric shapes are also possible. In addition, the signal-processing control unit 6 is shown schematically in FIG. 1 and FIG. 2.

The measuring chamber sample Pr to be analyzed, which in the embodiment comes from the breath sample A, flows through an inlet-side opening Ö.e into the interior of the measuring chamber 3, e.g. by being drawn in and/or diffusing into the measuring chamber 3. In one embodiment, the measuring chamber sample Pr flows back out of the measuring chamber 3 through an outlet-side opening Ö.a. Thanks to this implementation, the sensor 12 can examine a plurality of measuring chamber samples Pr in rapid succession. It is also possible that an outlet-side opening Ö.a is not provided, and the measuring chamber sample Pr flows back out of the measuring chamber 3 through the inlet-side opening Ö.e.

The electrochemical sensor 12 comprises
a measuring electrode 20 which is electrically contacted by a contact wire 34,
a counter-electrode 21 which is electrically contacted by a contact wire 33,
an ionically conductive electrolyte 28 between the two electrodes 20 and 21,
a connecting wire 22 which electrically connects the two contact wires 33 and 34 and comprises an electrical measuring resistor 29,
optionally a reference electrode (not shown), and
a current intensity sensor 38 which repeatedly measures the current intensity I of the current flowing through the connecting wire 22.

The electrolyte 28 is preferably provided by a membrane. Such an electrochemical sensor 12 is also referred to below as a membrane electrode electrolyte unit (MPEU).

The electrolyte 28 is an electrically conductive medium, for example sulfuric acid or phosphoric acid or perchloric acid diluted with water. In the electrolyte 28, ions can move as electrical charge carriers, thus producing electrical conductivity. Preferably, a porous membrane provides the electrolyte 28. The electrolyte 28 establishes an ionically conductive connection between the measurement electrode 20 and the counter-electrode 21.

The control unit 6 receives a signal from the current intensity sensor 38 which describes the measured current intensity I=I(t).

In one embodiment, a temperature sensor 52 measures the temperature in the environment of the sensor arrangement 50. The control unit 6 receives a signal from the temperature sensor 52 which describes the measured ambient temperature.

The sensor 12 is configured such that the measuring chamber sample Pr only reaches the measuring electrode 20 and not the counter-electrode 21. In the example shown, the measuring electrode 20 is located on a wall of the measuring chamber 3, and the wall 40 and the electrolyte 28 prevent a significant amount of the measuring chamber sample Pr from reaching the counter-electrode 21.

The two contact wires 33 and 34 are electrically conductive and consist of a material which is not chemically attacked by the electrolyte 28, for example platinum or gold. The electrodes 20 and 21 also consist of a chemically resistant material, for example likewise platinum or gold. In many cases, the chemically resistant material of the electrodes 20, 21 also acts as a catalyst for a chemical reaction that is caused by the target gas, in this case breath alcohol, and is used for the measurement.

In one implementation, the electrochemical sensor 12 operates on the principle of a fuel cell. The measuring electrode 20 and the electrolyte 28 adsorb the target gas, for example ethanol, in the measuring chamber sample Pr. The adsorbed ethanol is then oxidized on the principle of the fuel cell. The chemical reaction used for the measurement therefore involves the step of oxidizing the breath alcohol in the measuring chamber sample Pr in the measuring chamber 3. Ideally, the entire amount of the breath alcohol in the measuring chamber sample Pr is oxidized.

As a result of the chemical reaction, an electric current I flows between the measuring electrode 20 and the counter-electrode 21 and thus through the connecting wire 22 comprising the measuring resistor 29. The current intensity sensor 38 measures the current I that varies over time. The control unit 6 derives the electrical charge Q, i.e. the total amount of electrical current flowing through the connecting wire 22 (principle of coulometry). As a rule, electric current flows until the entire electrochemically oxidizable gas, in this case the entire breath alcohol present in the measuring chamber 3, has actually been electrochemically converted. For a given volume of measuring chamber sample Pr in the measuring chamber 3, the more breath alcohol the measuring chamber sample Pr contains, before said breath alcohol is electrochemically converted, the higher the measured electrical charge Q will be. The measured electrical charge Q is therefore an indicator (a measure) for the breath alcohol content in the measuring chamber sample Pr and thus of the alcohol content in the subject's blood.

The control unit 6 applies a given relationship in order to derive the sought concentration (con) of breath alcohol in the measuring chamber sample Pr from the measured charge Q. For example:

$$con = F ( Q ) , \qquad ( 1 )$$

$$con = α * Q . \qquad ( 2 )$$

This relationship and other relationships described below are set up in advance in a computer-executable form and are stored in a computer-evaluable form in the data memory 7. The control unit 6 has, at least from time to time, read access to the data memory 7.

FIGS. 3A and 3B show four different current intensity curves as examples and illustrate the dependency on the electrolyte humidity Ef and on the ambient temperature. The time is plotted on the x-axis, the current intensity I on the y-axis. I[Ef, Temp](t) denotes the current intensity curve at an electrolyte humidity Ef and an ambient temperature Temp. In all four curves, the same concentration of breath alcohol is present in the analyzed gas sample.

In this example, the possible electrolyte humidity Ef is coded with a value from the value range of −1 (minimum electrolyte humidity) to +1 (maximum electrolyte humidity). It is not necessary to measure the actual electrolyte humidity. In FIG. 3A, the electrolyte humidity Ef assumes the value 0, i.e. an average value. The ambient temperature is −5° C. or +45° C. At a high ambient temperature, a high maximum current intensity Iₘₐₓ is achieved, but the current intensity decreases very quickly. In both curves, the area under the curve is approximately the same, meaning that the same amount of combustible target gas has been oxidized and the same electrical charge Q has flowed.

In FIG. 3B, the ambient temperature is +20° C. The electrolyte humidity assumes the value +0.95 or −0.95, which is very low (relatively dry electrolyte) or very high (relatively humid electrolyte). At a high electrolyte humidity Ef, similar to a high ambient temperature, a high maximum current intensity Iₘₐₓ is achieved, but the current intensity I rapidly decreases again. In addition, at a high electrolyte humidity Ef, the electrical charge Q and thus under the area under the curve is larger than in the case of a low electrolyte humidity Ef.

FIG. 4 and FIG. 5 show an exemplary current intensity curve achieved during the analysis of the measuring chamber sample Pr. The time t is plotted on the x-axis, and the current intensity I(t) in [mA] measured at each time point t is plotted on the y-axis. FIG. 4 and FIG. 5 thus show, by way of example, a typical current intensity curve (current measurement curve) I(t). This current intensity curve I(t) occurs when the measuring chamber sample Pr contains breath alcohol. The current intensity I typically increases from the beginning of the measurement to a maximum Iₘₐₓ and then decreases again.

As is known, the area under the curve I(t) from t=0 to a time point t>0 is equal to the electrical charge Q that has flowed up to the time point T.

The control unit 6 determines the electrical charge Q from the current intensity curve I(t). The electrical charge Q serves as the detection variable for the breath alcohol content. The measured electrical charge Q depends on the desired breath alcohol content in the breath sample A and thus in the measuring chamber sample Pr, and in addition on the following variables:
the electrolyte humidity Ef,
the humidity of the measuring chamber samplePr,
the temperature of the electrodes 20 and 21, and
the ambient temperature Temp.

In many cases, a predefined standard value can be used for the humidity of the breath sample A and thus of the measuring chamber sample Pr. A predefined standard value can often also be used for the ambient temperature. It is also possible for the control unit 6 to receive and process a measured value for the current ambient temperature.

In one embodiment, the analyzer 100 comprises a sensor that measures the temperature of the electrodes 20 and 21. Often, the electrode temperature may assume a linear influence on the electrical charge Q. In another embodiment, a standard value or reference value is used for the electrode temperature.

In the following, Qₘₑₐₛ denotes the measured electrical charge Q, optionally adjusted by the influence of the humidity of the measuring chamber sample Pr, the electrode temperature and/or the ambient temperature.

The way in which the influence of the electrolyte humidity Ef on the measured charge Qₘₑₐₛ is taken into account is described below. The humidity of the electrolyte 28 is influenced by environmental conditions, in particular by the ambient humidity. Inevitably, the measuring chamber 3 and thus the electrolyte 28 to be specific are at least temporarily fluidically connected to the environment, namely at least when the subject gives a breath sample A and a portion of the breath sample A flows as the measuring chamber sample Pr into the measuring chamber 3. In addition, the electrolyte humidity Ef usually decreases when the analyzer 100 is stored for a relatively long period of time in an environment of relatively low relative humidity.

As a rule, at a constant target gas concentration, the higher the electrolyte humidity Ef is, the greater is the measured electrical charge Qₘₑₐₛ. For this reason, the control unit 6 applies a computer-evaluable concentration function

$$con = F [ Ef ] ( {Q}_{m e a s} ) \qquad ( 3 )$$

which takes into account the effect of the electrolyte humidity Ef.

At a constant concentration con of the target gas, here breath alcohol, the higher the electrolyte humidity Ef, the greater the charge Qₘₑₐₛ. For this reason,

$${Q}_{m e a s} = G ( Ef ) * {Q}_{ref} . \qquad ( 4 )$$

Here, Q_{ref} is a reference value for the electrical charge Q that occurs under given reference conditions, such as at a certain target gas concentration, an electrolyte humidity Ef of 50% (0 in the range from −1 to +1) and a certain reference temperature. The ambient humidity can affect the electrolyte humidity Ef, but usually does not directly influence the electrical charge Qₘₑₐₛ to a significant extent. In many cases, it is justified to assume that at a constant concentration con of breath alcohol, a linear relationship between the electrical charge Q and the electrolyte humidity Ef in the form of

$$G ( Ef ) = a + b * Ef , i . e . {Q}_{m e a s} = ( a + b * Ef ) * {Q}_{ref} , \qquad ( 5 )$$

describes the dependency with a sufficient degree of accuracy.

FIG. 4 and FIG. 5 show different parameters of the current intensity curve I(t). FIG. 4 shows, among other things:
the maximum current intensity Iₘₐₓ,
the inflection point Wp, at which the curvature of the current intensity curve I(t) changes, and also the time point t_{Wp} at which this inflection point Wp occurs,
the time period Tₘₐₓ, which extends between the start t=0 of measurement and the time point tₘₐₓ at which the current flows with maximum current intensity Iₘₐₓ,
a partial integral Qₖ, which is the charge flowing between two time points t₁ and t₂, i.e. the area under the current intensity curve I(t) between these two time points t₁ und t₂, where, for example, the time point t_{Wp} of the inflection point Wp lies between the two time points t₁ and t₂ or wherein, at the time point t₁ the current intensity is x₁% of the maximum current intensity for the first or last occurrence and, at the time point t₂ is x₂% for the first or last occurrence,
the time period T_{a,b}, which occurs between two characteristic time points tₐ and t_{b} of the current intensity curve I(t),
the change I′(t_{b}) of the current intensity I(t) at a certain time point to, i.e. the slope of the current intensity curve I(t) at the time point t_{b}, and
the slope of the secant Sk between two characteristic time points tₐ und t_{b}, i.e. [I(t_{c})−I(tₐ)]/(t_{c}−tₐ).

FIG. 5 shows the time points at which the current intensity curve I(t) reaches 30%, 35%, 40%, 70% and 100% of the maximum current intensity Iₘₐₓ. A plurality of time periods is shown, for example the time period T_{70.70}, which extends between the following two time periods:
the time point t_{i,70}, at which the current intensity I becomes greater than 70% of the maximum value Iₘₐₓ for the first time, and
the time point t_{d,70}, where it falls below 70% again for the first time.

In addition, the time period T₇₀ is plotted which period extends from the beginning of measurement (t=0) until the current intensity I reaches 70% of the maximum value Iₘₐₓ. In addition, the time period T_{standard} is plotted, which is the time period that extends between the following two time points:
the time point t_{i,40}, at which the current intensity curve I(t) becomes for the first time greater than 40% of the maximum value Iₘₐₓ, and
the time point t_{d,30}, at which the current intensity curve I(t) falls again below 30% of the maximum value Iₘₐₓ.

These curve parameters are intended to be understood as examples only.

FIG. 4 and FIG. 5 thus show, for example, M+N different curve parameters X₁, . . . , X_{M}, Y₁, . . . , Y_{N} of the current intensity curve I(t). After the control unit 6 has determined the current intensity curve I(t), a value x₁, . . . , x_{M} can be derived for each curve parameter X₁, . . . , Xₘ and a value y₁, . . . , y_{N} can be derived for each gradient parameter Y₁, . . . , Y_{N}. In one embodiment the electrolyte humidity Ef is calculated according to the humidity function

$$Ef = [ {a}_{1} * {x}_{1} + … + {a}_{M} * {x}_{M} ] / [ {b}_{1} * {y}_{1} + …o + {b}_{N} * {y}_{N} ] \qquad ( 6 )$$

The weighting factors a₁, . . . , a_{M}, b₁, . . . , b_{N} are determined in advance. In another embodiment, a neural network is trained in advance using a sample. The curve parameters X₁, . . . , X_{M}, Y₁, . . . , Y_{N} are input variables of this neural network.

In internal tests, the inventors generated in advance a sample. To generate the sample, they used a calibration apparatus (not shown) that comprises sensors that are needed only for generating the sample but not for operating the analyzer 100. This calibration apparatus can be connected to an electrochemical sensor 12, in particular to an electrochemical sensor 12 which is constructed as shown in FIG. 2. The calibration apparatus receives a signal from the corresponding current intensity sensor 38, which belongs to a connected electrochemical sensor 12.

In one embodiment, the calibration apparatus comprises a climate cabinet with a climate chamber. In this climate chamber, a given ambient temperature and a given ambient humidity can be set. An electrochemical sensor 12 can be placed in this climate chamber. The electrolyte humidity Ef of a sensor 12 in the climate chamber corresponds to the ambient humidity in the climate chamber after a settling time period. Preferably, the climate chamber is free of the or of any target gas to be detected.

Various electrochemical sensors 12 are placed in the climate chamber one after the other. For each sensor 12, at least two different ambient humidities and preferably also at least two different ambient temperatures are set one after the other. Each sensor 12 provides one sample element for each ambient humidity and each ambient temperature. This sample element includes an electrolyte humidity Ef, namely the set ambient humidity, and the resulting current intensity curve I(t).

The sample is evaluated with the following two objectives:
The electrolyte humidity Ef should be determinable with a measurement error of less than 10%.
The estimated value for the electrolyte humidity Ef is to have relatively little dependence on the ambient temperature.

In particular, suitable curve parameters X₁, . . . , X_{M}, Y₁, . . . , Y_{N} were identified. The internal test produced the following results:

A good estimated value of the electrolyte humidity Ef can be obtained if the four curve parameters T₇₀, T_{70,35}, T_{70,70} and T_{standard} from FIG. 5 are used.

The following formula was empirically discovered to be a good humidity function Ef:

$$Ef = [ {a}_{1} * {T}_{70} + {a}_{2} * {T}_{70 , 70} + {a}_{3} * {T}_{70 , 35} ] / {T}_{standard} . \qquad ( 7 )$$

The three weighting factors a₁, a₂, a₃ are determined empirically, and namely preferably using the sample described above. Preferably, the three weighting factors a₁, a₂, a₃ are determined beforehand such that the difference between the actual electrolyte humidity, i.e. the humidity in the climate chamber, and the electrolyte humidity determined according to the humidity function (7) is minimized. Ef is a coded value between −1 and +1.

FIG. 6 shows exemplary measurement results. The time is plotted on the x-axis and the electrolyte humidity Ef determined is plotted on the y-axis. Just as in FIGS. 3A and 3B, the electrolyte humidity Ef is coded with a value from the range of −1 (lowest possible electrolyte humidity, occurs at the lowest possible ambient humidity of 0%) to +1 (highest possible electrolyte humidity, occurs at the highest possible ambient humidity of 100%). To determine this coded value, formula (7) was used. The actual electrolyte humidity was changed, and namely in particular by changing the ambient humidity. As already explained, low ambient humidity leads to low electrolyte humidity after a settling time period, and high ambient humidity leads to high electrolyte humidity. Other environmental conditions have a significantly smaller influence on actual electrolyte humidity. To take this settling time period into account, the electrolyte humidity Ef was determined once a day. Within one day, the actual electrolyte humidity assumes a value that is determined by the ambient humidity. On days 1 to 6, the ambient humidity and thus the actual electrolyte humidity were increased from −1 (minimum value) to +1 (maximum value) and on days 7 to 10 reduced again to the minimum value of −1. On each day, one respective measurement was performed for three different ambient temperatures, namely on days 1 to 6, a respective measurement at +15° C., +25° C. and +35° C. and, on days 7 to 10, a respective measurement at −5° C., +20° C. and +45° C. In total, three measurements per day were performed.

To determine the electrolyte humidity Ef, formula (7) given above was applied with previously empirically determined weighting factors a₁, a₂, a₃. Ef [Temp] denotes the determined coding of the electrolyte humidity Ef at the ambient temperature Temp. Ef [Temp] is therefore a number from the range [−1, +1].

The following results were achieved:
Each determined electrolyte humidity Ef differed from the actual electrolyte humidity at each ambient temperature Temp by a maximum of plus or minus 15%. More precisely: the coding for the electrolyte humidity determined according to the disclosure (a value between −1 and +1) differed by a maximum of 15% from the electrolyte humidity that occurs at the set ambient humidity. This is a sufficient degree of accuracy for several applications.
As can be seen in FIG. 6, the ambient temperature Temp has a relatively small effect on the determined electrolyte humidity Ef. The effect of the ambient temperature on the determination result could therefore be largely eliminated by calculation.

As already explained, in one embodiment the electrochemical sensor 12 in FIG. 2 is a component of an analyzer 100. The analyzer 100 guides a measuring chamber sample Pr into the measuring chamber 3, and the electrochemical sensor 12 analyzes this measuring chamber sample Pr. The control unit 6 determines the current intensity curve I(t) shown by way of example in FIG. 4 and FIG. 5. The control unit 6 deduces the total electrical charge Q=Qₘₑₐₛ and uses the charge Qₘₑₐₛ to decide whether or not the measuring chamber sample Pr contains breath alcohol above a predetermined lower concentration limit. Knowledge of the electrolyte humidity Ef is not required for this decision.

In one embodiment, the detection of the event in which the measuring chamber sample Pr contains breath alcohol above the concentration limit triggers the step of issuing a corresponding notification in at least one form that can be perceived by a person.

The detection process just described also triggers the following steps: The control unit 6 determines the current humidity Ef of the electrolyte 28. For example, the control unit 6 determines the value of a plurality of curve parameters of the current intensity curve I(t) and applies a humidity function, for example humidity function (6). The predetermined weighting factors a₁, a₂, a₃ are part of a program which the control unit 6 applies or are stored in the data memory 7 of the analyzer 100.

At a constant concentration con of the target gas, for example breath alcohol, the higher the electrolyte humidity Ef is, the greater is the measured charge Qₘₑₐₛ; cf. calculation rule (4). This dependency can be described by a functional relationship. For example, linear relationship (5) is applicable with a sufficient degree of accuracy. The control unit 6 uses the determined electrolyte humidity Ef to compensate for the influence of the electrolyte humidity Ef on the measurement. Preferably, the control unit 6 deduces a corrected charge Q_{corr}, specifically according to calculation rule

$${Q}_{c o r r} = {G}^{- 1} ( Ef ) * {Q}_{m e a s} \qquad ( 8 )$$

G⁻¹ is the inverse function of the function G=G (Ef) in calculation rule (4). The desired target gas concentration con is determined by applying relationship F in calculation rule (1) to the corrected voltage Q_{corr}. For example, the following applies:

$$con = α * {Q}_{c o r r} . \qquad ( 9 )$$

In addition, the control unit 6 detects the undesirable event in which the electrolyte humidity Ef lies outside a given target value range for a sufficiently long period of time. This event may be an indication that electrolyte 28 has evaporated and the sensor 12 is no longer able to generate reliable measurement results despite the calculation-based compensation just described. Preferably, the control unit 6 causes a notification containing this event to be generated and output in at least one form perceptible by a person.

### LIST OF REFERENCE SIGNS

| | |
|---|---|
| 1 | Mouthpiece, can be detachably attached to the tube 2 |
| 2 | Tube, can be detachably connected to the housing 4, |
| | carries the mouthpiece 1 |
| 3 | Measuring chamber, accommodates the electrochemical |
| | sensor 12 |
| 4 | Housing, accommodates the measuring chamber 3, the |
| | sensor 12, the control unit 6, and the data memory 7, carries |
| | the tube 2 |
| 6 | Control unit, receives and processes a signal from the |
| | current intensity sensor 38 and optionally a signal from the |
| | temperature sensor 52, determines the current intensity |
| | curve I(t), the electrical charge Q_{meas,} and the electrolyte |
| | humidity Ef |
| 7 | Data memory to which the control unit 6 has at least from |
| | time to time read access |
| 12 | Electrochemical sensor 12, comprises the electrodes 20 |
| | and 21, the contact wires 33 and 34 and the electrolyte 28 |
| 20 | Measuring electrode, electrically contacted by the contact |
| | wire 34 |
| 21 | Counter-electrode, electrically contacted by the contact wire |
| | 33 |
| 22 | Connecting wire between the contact wires 33 and 34 |
| 28 | Electrolyte between the two electrodes 20 and 21, has the |
| | humidity Ef |
| 29 | Electrical measuring resistor in the connecting wire 22 |
| 33 | Contact wire for the counter-electrode 21 |
| 34 | Contact wire for the measuring electrode 20 |
| 38 | Current intensity sensor, measures the intensity of the |
| | current flowing through the connecting wire 22 |
| 40 | Wall for the measuring chamber 3 |
| 50 | Sensor arrangement, comprises the electrochemical sensor |
| | 12, the measuring chamber 3 and the wall 40 |
| 52 | Optional temperature sensor, measures the temperature in |
| | the environment of the sensor arrangement 50 |
| 100 | Analyzer, comprises the housing 4, the sensor arrangement |
| | 50 having the electrochemical sensor 12 and the measuring |
| | chamber 3, the control unit 6, the data memory 7, the tube |
| | 2 and the mouthpiece 1 as well as a power supply unit (not |
| | shown) |
| A | Breath sample, is given into the mouthpiece 1, flows |
| | through the tube 2 |
| Ef | Current humidity of the electrolyte 28, is determined by the |
| | control unit 6 |
| Ef[Temp] | Determined electrolyte humidity at the ambient temperature |
| | Temp |
| G | Functional relationship, provides the electrical charge as a |
| | function of the electrical charge at a reference electrolyte |
| | humidity and the actual electrolyte humidity |
| I(t) | Temporal curve of the intensity I of the current flowing |
| | through the connecting wire 22, is determined on the basis |
| | of a signal from the current intensity sensor 38 |
| I([Ef, | Temporal curve of the current intensity I at an electrolyte |
| Temp](t) | humidity Ef and an ambient temperature Temp |
| Iₘₐₓ | Maximum value of the current intensity curve I(t), is |
| | assumed at the time point tₘₐₓ |
| I′(t_{Ip}) | Temporal change of the current intensity I at the time point |
| | t_{Ip} |
| MA | Central axis of the wall 40 |
| Ö.a | Outlet-side opening from the measuring chamber 3 |
| Ö.e | Inlet-side opening into the measuring chamber 3 |
| Pr | Measuring chamber sample, is diverted from the breath |
| | sample A which flows through the tube 2, enters the |
| | measuring chamber 3, may contain breath alcohol as the |
| | target gas to be detected |
| Qₖ | Electric charge flowing between the time points t₁ and t₂ |
| Q_{corr} | Value for the electrical charge Qₘₑₐₛ corrected by using the |
| | electrolyte humidity Ef determined |
| Qₘₑₐₛ | Electric charge derived from the measured current intensity |
| | curve I(t) |
| Q_{ref} | Reference value for the electrical charge Q, is achieved |
| | under given reference conditions, in particular in the case of |
| | a reference value for the electrolyte humidity Ef |
| Sk | Secant between the two points [tₐ, I(tₐ)] and t_{b}, I(t_{b})] |
| t_{d, y} | Latest time point at which the current intensity I(t) is y % of |
| | the maximum current intensity Iₘₐₓ |
| Temp | Ambient temperature |
| t_{i, x} | Earliest time point at which the current intensity curve I(t) is |
| | x % of the maximum current intensity Iₘₐₓ |
| tₘₐₓ | Time point in at which the current intensity curve I(t) |
| | assumes the maximum value Iₘₐₓ |
| t_{Ip} | Time point at which the current intensity curve I(t) comprises |
| | the inflection point Wp and the slope I′(t_{Wp}) |
| Tₓ | Time period between the start of the measurement (t = 0) and |
| | the time point t_{i, x} |
| T_{x, y} | Time period between the two time points t_{i, x} and t_{d, y} |
| T_{standard} | Time period between the two time points t_{i, 40} and t_{d, 30} |
| Wp | Inflection point of the current intensity curve I(t), is assumed |
| | at the time point t_{Wp} |

1. A test device for testing an electrochemical sensor;
the electrochemical sensor comprising a measuring chamber configured to take up a gas sample, a first electrode, a second electrode, and an ionically conductive electrolyte positioned between the first and second electrodes, the electrolyte having a humidity that varies over time;
wherein a target gas, as a component of a gas sample in the measuring chamber, induces an electrochemical reaction which causes an electric current to flow between the first and second electrodes;
wherein a plurality of curve parameters and a computer-evaluable humidity function are provided;
wherein each curve parameter is a parameter of a time course of a current intensity;
wherein the computer-evaluable humidity function describes the humidity of the electrolyte as a function of the curve parameters; and
wherein the test device comprises a control unit configured to determine a current intensity curve, the current intensity curve being the time course of the intensity of the current caused to flow by the electrochemical reaction and having a maximum current intensity; and
wherein the control unit is further configured to determine a current humidity of the electrolyte by determining,
wherein, for determining the current humidity (Ef) of the electrolyte (28), the control unit (6) is designed to determine, for each curve parameter, a value that the curve parameter assumes for the current intensity curve and by applying the computer-evaluable humidity function to the parameter values.

2. The test device according to claim 1 wherein,
at least one curve parameter is:
a time period that extends between a start of a measurement and an earliest or latest time point at which the current intensity curve is equal to x₁% of the maximum current intensity; or
a time period that extends between the earliest or latest time point, at which the current intensity curve is equal to x₁% of the maximum current intensity, and an earliest or latest time point, at which the current intensity curve is equal to x₂% of the maximum current intensity, wherein x₁ and x₂ are percentages that coincide or differ from each other; or
an electrical charge that flows between a start of a measurement and an earliest or latest time point at which the current intensity curve is equal to x₁% of the maximum current intensity; or
an electrical charge that flows between an earliest or latest time point, at which the current intensity curve is equal to x₁% of the maximum current intensity, and the earliest or latest time point, at which the current intensity curve is equal to x₂% of the maximum current intensity; or
a slope of a secant between a start of a measurement and an earliest or latest time point at which the current intensity curve is equal to x₁% of the maximum current intensity; or
an earliest or latest time point, at which the current intensity curve is equal to x₁% of the maximum current intensity and an earliest or latest time point, at which the current intensity curve is equal to x₂% of the maximum current intensity.

3. The test device according to claim 1 wherein a curve parameter of the current intensity curve is the time span between the earliest time point at which the current intensity curve is equal to x₁% of the maximum current intensity, and the latest time point at which the current intensity curve is equal to x₂% of the maximum current intensity, wherein x₁ and x₂ are two percentages that coincide or differ from each other;
wherein the humidity function is a quotient of a numerator humidity function and a denominator humidity function;
wherein the time span between the earliest and the latest time point occurs in the denominator humidity function; and
wherein at least one other curve parameter of the current intensity curve occurs in the numerator humidity function.

4. The test device according to claim 3 wherein characterized in that
the denominator humidity function is equal to the time span between the earliest and the latest time point, and the numerator humidity function is a weighted average of at least two other curve parameters of the current intensity curve.

5. The test device according to claim 1 wherein characterized in that
the control unit is configured to receive a signal from a temperature sensor;
wherein the signal describes an ambient temperature in an environment of the electrochemical sensor measured by the temperature sensor; and
wherein the humidity function describes the humidity of the electrolyte as a function of the curve parameters and an ambient temperature.

6. An analyzer comprising:
a measuring chamber;
a first electrode;
a second electrode;
an ionically conductive electrolyte between the first and second electrodes; and
a control unit;
wherein the electrolyte has a humidity which varies over time;
wherein the measuring chamber is configured to take up a gas sample;
wherein a target gas, as a component of a gas sample in the measuring chamber, induces an electrochemical reaction which causes an electric current to flow between the first and second electrodes;
wherein a detection variable of the electric current correlates with a concentration of the target gas in the gas sample in the measuring chamber;
wherein the control unit is configured to measure the concentration of the target gas based on the detection variable of the electric current;
wherein a plurality of curve parameters and a computer-evaluable humidity function are provided;
wherein each given curve parameter is a parameter of a time course of a current intensity;
wherein the computer-evaluable humidity function describes a humidity of the electrolyte as a function of the curve parameters;
wherein the control unit is further configured to determine a current intensity curve, that is the time course of the intensity of the current caused to flow by the electrochemical reaction;
wherein the control unit is further configured to determine a current humidity of the electrolyte by using the current intensity curve;
wherein the control unity is further configured to determine the current humidity of the electrolyte by determining, for each given curve parameter, a value that the curve parameter assumes for the current intensity curve and to applying the humidity function to the parameter values.

7. The analyzer according to claim 6 wherein a concentration function is given in a computer-evaluable form;
wherein the concentration function describes the target gas concentration as a function of the detection variable and of the electrolyte humidity, and
wherein the control unit is configured to measure the target gas concentration by applying the given concentration function to the detection variable and the electrolyte humidity.

8. The analyzer according to claim 6 wherein the control unit is configured to decide, depending on the detection variable of the electric current, whether or not the gas sample contains the target gas at a concentration within or outside a given concentration value range, and to determine the electrolyte humidity at least when the target gas concentration is outside the given concentration value range.

9. A test method comprising:
providing an electrochemical sensor comprising a measuring chamber configured to take up a gas sample, a first electrode, a second electrode, and an ionically conductive electrolyte between the first and second electrodes, the electrolyte having a humidity that varies over time;
providing in a computer-evaluable form a plurality of curve parameters and a humidity function;
wherein each curve parameter is a parameter of a time course of a current intensity and
wherein the computer-evaluable humidity function describes the humidity of the electrolyte as a function of the curve parameters;
determining a current intensity curve, the current intensity curve being a time course of an intensity of a current, the current flowing between the first and second electrodes and being caused by an electrochemical reaction from a target gas in a measuring chamber, the current intensity curve having a maximum current intensity; and
determining a current humidity of an electrolyte positioned between a first and second electrode in the measuring chamber by using the current intensity curve;
wherein the step of determining the current humidity comprises determining for each of the plurality of curve parameters, which value the curve parameter assumes for the current intensity curve and applying the computer-evaluable humidity function to the parameter values.

10. The test method according to claim 9 wherein
at least one curve parameter of the current intensity curve is:
a time period between a start of a measurement and an earliest time point at which the current intensity curve is equal to x₁% of the maximum current intensity; or
a time period between an earliest or latest time point at which the current intensity curve is equal to x₁% of the maximum current intensity and an earliest or latest time point at which the current intensity curve is equal to x₂% of the maximum current intensity,
wherein x₁ and x₂ are two percentages that are the same or differ; or
an electrical charge which flows between
an earliest or latest time point at which the current intensity curve is equal to x₁% of the maximum current intensity and an earliest or latest time point at which the current intensity curve is equal to x₂% of the maximum current intensity; or
a slope of a secant between an earliest or latest time point, at which the current intensity curve is equal to x₁% of the maximum current intensity, and an earliest or latest time point, at which the current intensity curve is equal to x₂% of the maximum current intensity.

11. A gas concentration measuring method comprising:
causing a gas sample to flow into a measuring chamber;
measuring a detection variable of an electric current flowing between a first electrode and a second electrode, an ionically conductive electrolyte being positioned between the first and second electrodes, the electric current being caused by an electrochemical reaction caused by a target gas, as a component of the gas sample, in the measuring chamber;
determining a current intensity curve, which is the time course of the intensity of the electric current, the detection variable of the electric current correlating with the concentration of the target gas;
determining a current humidity of the electrolyte by determining, for each of a plurality of given curve parameters, which value the curve parameter assumes for the current intensity curve and applying a given computer-evaluable humidity function to the values.

12. The measuring method according to claim 11, wherein a concentration function is given in a computer-evaluable form;
wherein the concentration function describes a concentration of the target gas as a function of the detection variable and of the electrolyte humidity; and
wherein measuring the target gas concentration comprises applying the given concentration function to the detection variable and the electrolyte humidity.

13. The measuring method according to claim 11 wherein characterized in that
the detection variable is used to decide whether the gas sample contains the target gas at a concentration within or outside a given concentration range;
wherein detecting that the gas sample contains the target gas at a concentration outside the given concentration range triggers the step of determining the current electrolyte humidity.

14. A device comprising:
an electrochemical sensor comprising a measuring chamber configured to take up a gas sample, a first electrode, a second electrode, and an ionically conductive electrolyte positioned between the first and second electrodes, the electrolyte having a humidity that varies over time; and
a test device comprising a control unit;
wherein a target gas, as a component of a gas sample in the measuring chamber, induces an electrochemical reaction which causes an electric current to flow between the first and second electrodes;
wherein a plurality of curve parameters and a computer-evaluable humidity function are provided;
wherein each curve parameter is a parameter of a time course of a current intensity;
wherein the computer-evaluable humidity function describes the humidity of the electrolyte as a function of the curve parameters; and
wherein the control unit configured to determine a current intensity curve, the current intensity curve being the time course of the intensity of the current caused to flow by the electrochemical reaction and having a maximum current intensity; and
wherein the control unit is further configured to determine a current humidity of the electrolyte by determining, for each curve parameter, a value that the curve parameter assumes for the current intensity curve and by applying the computer-evaluable humidity function to the parameter values.
